SEQUENCE LISTING (9 sequences)

SEQ ID NO: 1 - protein, 27 aa, Homo sapiens
  VARIANT (15): Disease marker sequence
EDASSDSTGADPAQZAGTSQPNEDIAG

SEQ ID NO: 2 - protein, 5 aa, Homo sapiens
  Disease marker sequence
WDPKE

SEQ ID NO: 3 - protein, 74 aa, Homo sapiens
  Disease marker sequence
DSGCKLLEDMVEKTINSDISIPEYKELLQEFIDSDAAAEAMGKFKQCFLNQSHRTLKNFGLMMHTVYDSIWCNL

SEQ ID NO: 4 - protein, 5 aa, Homo sapiens
  N terminus of disease marker sequence
EDASS

SEQ ID NO: 5 - protein, 5 aa, Homo sapiens
  N terminus of disease marker sequence
SSSKE

SEQ ID NO: 6 - protein, 13 aa, Homo sapiens
  N terminus of disease marker sequence
DSGCKLLEDMVEK

SEQ ID NO: 7 - protein, 9 aa, Homo sapiens
  N terminus of disease marker sequence
HHLLASDEE

SEQ ID NO: 8 - protein, 4 aa, Homo sapiens
  N terminus of disease marker sequence
SDEE

SEQ ID NO: 9 - protein, 4 aa, Homo sapiens
  N terminus of disease marker sequence
GRRR

(19) United States
(12) Patent Application Publication
(10) Pub. No.: US 2005/0124011 A1
(43) Pub. Date: Jun. 9, 2005

(54) Diagnosis of disease using tears

(75) Inventors: Carol Morris, New South Wales (AU); Mark Willcox, New South Wales (AU); Shirley Bolis, Griffith (AU); Bradley Walsh, New South Wales (AU); Ben Herbert, New South Wales (AU); Mark Molloy, Ann Arbor, MI (US); Andrew Arthur Gooley, New South Wales (AU); Keith Leslie Williams, New South Wales (AU)

Correspondence Address: MORGAN LEWIS & BOCKIUS LLP, 1111 PENNSYLVANIA AVENUE NW, WASHINGTON, DC, 20004, US

(73) Assignees: Macquarie Research Limited; Unisearch Limited

(21) Appl. No.: 10/896,868

(22) Filed: Jul. 23, 2004

Related U.S. Application Data
(63) Continuation of application No. 09/367,009, filed on Nov. 8, 1999.

(30) Foreign Application Priority Data
Feb. 7, 1997 (AU) PO5009

(51) Int. Cl. G01N 33/53; G01N 33/574; C07K 14/47

(52) U.S. Cl. 435/7.23; 530/350

(57) ABSTRACT

A method of screening or detecting non-ocular disease in an animal, the method comprising the steps of: (a) obtaining a tear sample from the animal; (b) separating biomolecules present in the tear sample; and (c) detecting for the presence or absence of one or more biomolecules such that the presence or absence of the one or more biomolecules being an indicator or marker of a disease state in the animal.

### TECHNICAL FIELD

The present invention relates generally to diagnosis of disease in animals including humans by detecting disease markers in tears.

### BACKGROUND ART

The early detection or prediction of disease in humans and animals is often important for initiating appropriate medical management of the disease. In diseases like cancer and genetic disorders, early detection can often result in successful control or treatment of these conditions. In order to carry out early detection of disease, screening methods that are simple to carry out and do not involve invasive procedures are desirable.

Presently, analyses of bodily fluids like blood, plasma, serum, urine and cerebrospinal fluid are used to screen for disease indicators. Apart from urine, these fluids require medical intervention to be obtained involving some discomfort and risk to the patient. Other bodily excretions and fluids, however, have not been considered to be of use for general screening for disease.

In traditional medicine there is a well established discipline know as Iridology whereby health/disease status is diagnosed by examination of the eye. Currently, examination of the eye or eye fluids (eg tears) has not been used to diagnose disease (other than specific eye disease) or malfunction in conventional medicine or veterinary practice.

The present inventors have made the surprising discovery that tears can be analysed to detect markers in the form of proteins that can be used as indicators of disease in animals.

### DISCLOSURE OF INVENTION

In a first aspect, the present invention consists in a method of screening or detecting non-ocular disease in an animal comprising obtaining a tear sample from the animal and analysing the tear sample for an indicator or marker of the non-ocular disease.

In a preferred embodiment, the present invention consists in a method of screening or detecting non-ocular disease in an animal comprising the steps:
(a) obtaining a tear sample from the animal;
(b) separating biomolecules present in the tear sample; and
(c) detecting for the presence or absence of one or more biomolecules such that the presence or absence of the one or more biomolecules being an indicator or marker of a disease state in the animal.

Preferably, the animal is a human subject. It will be appreciated, however, that the present invention is also suitable for veterinary situations.

The non-ocular disease may be any disease that can be detected by the presence of a biological marker excreted with, or found in, tears. It will be appreciated that such diseases would include cancer and genetic disorders. Such diseases include breast and prostate cancer.

In a preferred embodiment, the disease marker is a protein, preferably a protein having a N-terminal sequence selected from:
EDASSDSTGA DPAQ(E/Q)AGTSQ PNEDIAG; WDPKE; and
DSGCKLLEDM VEKTINSDIS IPEYKELLQE FIDSDAAAEA MGKFKQCFLN QSHRTLKNFG LMMHTVYDSI WCNL.

More preferably, the disease marker is the protein:
DSGCKLLEDM VEKTINSDIS IPEYKELLQE FIDSDAAAEA MGKFKQCFLN QSHRTLKNFG LMMHTVYDSI WCNL, or part thereof.

In a further preferred form, the marker is a protein or proteins detected by separating the tear sample by a chromatographic technique. It will be appreciated that after separation, proteins can be identified by labelling the proteins using direct or indirect techniques known to the art. Any type of electrophoresis can be used, however, two-dimensional polyacrylamide gel electrophoresis (2D-PAGE) is particularly suitable to separate proteins from tear fluids. Protein banding and/or spot patterns from analyses of tears from patients and normal individuals using this method can be compared to detect abnormalities or differences. As particular protein markers will appear in known positions in a 2D-PAGE gel, for example, the presence or absence of a protein marker in a tear sample may be indicative of a disease state or a potential problem for the patient. Furthermore, a change in the relative position of a marker after the chromatography may indicate changes in that protein from the patient. This may also be relevant in diagnosis of disease as it may indicate a mutation or change in the protein or modifications which can cause or lead to a disease state.

It will be appreciated that other methods presently available to detect proteins in biological samples would also be useful to analyse tear samples for the presence of disease markers. Suitable methods include direct testing of a tear sample (neat or in a concentrated form, for example) using labelled probes in an immunoassay or radioimmunoassay. Antibodies can be prepared against proteins found in tears that are markers for disease states and used in such assays. The important discovery that tears may include protein markers indicative of a disease state will allow such assays to be developed.

The detection of one or more disease markers in a tear sample from a patient may alert the physician of a predisposition for disease in that patient. The present invention may be used to screen patients in a high risk group for a particular disease or be used to place patients in a particular risk group. The patient may be monitored further by other more invasive methods to confirm diagnosis or appropriate treatment can be started.

In a second aspect, the present invention consists in a protein marker detectable in tears having a N-terminus selected from:
EDASSDSTGA DPAQ(E/Q)AGTSQ PNEDIAG; WDPKE; and
DSGCKLLEDM VEKTINSDIS IPEYKELLQE FIDSDAAAEA MGKFKQCFLN QSHRTLKNFG LMMHTVYDSI WCNL.

Preferably, the protein marker detectable in tears includes the amino acid sequence:
DSGCKLLEDM VEKTINSDIS IPEYKELLQE FIDSDAAAEA MGKFKQCFLN QSHRTLKNFG LMMHTVYDSI WCNL, or part thereof.

It will be appreciated that antibodies may be generated to the protein markers according to this invention which could be used to detect the proteins in tears by suitable assays known to the art.

In a third aspect, the present invention consists in isolated nucleic acid molecules encoding the protein markers according to the second aspect of the present invention.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

In order that the present invention may be more clearly understood, preferred forms will be described with reference to the following examples.

### MODES FOR CARRYING OUT THE INVENTION

Methods

Sample Preparation

Reflex tears were collected using glass microcapillary tubes from 12 non-contact lens wearing male and female humans and pooled. Reflex tears were stimulated by gently rubbing the nasal mucosa with a cotton wool tipped bud. Care was taken to minimise ocular surface contact during the collection. As an added precaution, examination of the ocular surface using slit lamp biomicroscopy and fluorescein was conducted on all subjects after tear collection to discount the possibility of ocular surface damage. Each sample was centrifuged at 2,000 g for 10 min to remove cell debris, then recovered and stored at −80° C. prior to 2D-PAGE.

Two different batches of tears from different groups of individuals were examined by analytical and preparative 2-dimensional electrophoresis.

Two-Dimensional Electrophoresis

For analytical gels a 7 microliter of sample was thawed and added to 500 microliter of rehydration solution (8M Urea, 4% CHAPS, 100 mM DTT and 40 mM Tris). The solution was introduced into a sealed 2 ml plastic tissue culture pipette containing a single 18 cm Immobiline DryStrip (covering the range approximately pH 3.5-10). The strip was rehydrated at room temperature for 24 hours.

Preparative gels were prepared in the same manner, although 250 microliter of sample was loaded. The sample was lyophilised before dissolving in the 500 microliter of rehydration solution. The first dimension, isoelectric focussing (IEF), was carried out using a Pharmacia Multiphor II with a DryStrip Kit; power was supplied using a Consort 5000 V power supply and cooling water at 20° C. was supplied by a Pharmacia Multitemp III. Table 1 gives the running conditions for the IEF. After IEF, the strips were stored at −80° C. until required for the second dimension.

TABLE 1
Running conditions for the first dimension IEF

| Phase | V | mA (max) | W (Max) | Time (h) | Vh |
|---|---|---|---|---|---|
| 1 | 300 | 1.0 | 5.0 | 5 | 1,500 |
| 2 | 1000 | 1.0 | 5.0 | 10 | 10,000 |
| 3 | 3500 | 1.0 | 5.0 | 10 | 35,000 |
| 4 | 5000 | 1.0 | 5.0 | <100 | <500,000 |

Prior to the second dimension, the IPG strips were equilibrated for 2×10 minutes. For the first 10 min, the strips were placed in 6 M urea, 2% SDS, 20% glycerol 0.375 M Tris/HCl, pH 8.8 and 2% DTT. For the second 10 min, that solution was discarded and replaced by 6 M urea, 2% SDS, 20% glycerol 0.375 M Tris/HCl, pH 8.8 and 2.5% iodoacetamide.

Second dimension gels were run using the Bio-Rad Protean II Multicell system. The gels were 1.5 mm thick, 8-18% T gradients, and were crosslinked with PDA at 2.5% C. The gel and anode buffers were 0.375 M tris/HCl, pH 8.8. Cathode buffer was 192 mM glycine/tris pH 8.3, 0.1% (w/v) SDS and 0.001% (w/v) Bromophenol blue. The equilibrated IPG strips were embedded on the top of the SDS-PAGE gel using molten 1% (w/v) agarose in cathode buffer. Pharmacia low molecular weight standards were loaded in a single lane at one side of each gel. Gels were run at 25 mA per gel overnight, until the Bromophenol blue front had traversed the gel.

The completed analytical 2D gels were stained with an ammoniacal silver stain. Preparative gels were transferred to PVDF by standard methods. Proteins in preparative gels were visualised by staining with 0.1% (w/v) Coomassie Blue in 40% (v/v) methanol, after transfer to PVDF membranes.

N-Terminal Sequencing

Protein spots were excised from the PVDF membrane and loaded into a membrane-compatible Hewlett-Packard cartridge. Sequencing was conducted with a Model G1005A (Hewlett-Packard, CA) sequenator.

Results

Table 2 shows the results of N-terminal sequencing of 10 spots from a single preparative 2D gel transferred to PVDF membrane and stained with Coomassie Blue. Eight of the spots gave N-terminal sequences, with only spot 5 N-terminally blocked. This blocked protein was identified as human Zn-alpha-2 Glycoprotein by amino acid analysis matching to the database. Five different proteins were identified on the basis of N-terminal sequencing, with two proteins being represented in two different spots (1 & 2 being the same protein: 3 & 4 being the same protein) and one protein, the lipocalin Von Ebner's Gland Protein, being present in both intact and a N-terminally processed form. The present inventors believe this is the first report of N-terminal processing of Von Ebner's Gland Protein.

In all, seven different proteins were observed. Four of these proteins are known: human Cystatin S (spots 3 & 4); Human Von Ebner's Gland protein (spots 11 & 12) Human Zn-alpha-2 Glycoprotein (spot 5); and human lactotransferrin (spot 14).

Three new proteins were identified by this method. One (spot 9) is a protein of 74 amino acids with an apparent SDS-PAGE mass of 8 kDa and a predicted mass of 8.506 Da and is closely related to the uteroglobins. The Asparagine at position 50, while in the N-glycosylation motif Asn-Gln-Ser, is not glycosylated. The Cys at positions 4, 47 and 72 were identified as the acrylamide adducts of reduced Cysteine.

Uteroglobins are potent inhibitors of phospholipase A2 and are proteins of approximately 70 amino acids that form antiparallel disulfide-linked dimers. Examples of members of the uteroglobins are human mammaglobin (Swiss-Prot Q13296) and rat prostatic steroid-binding protein C3 (Swiss Prot P02780). These proteins are putative markers for breast and prostate cancer, respectively. Based on these similar proteins, the present inventors propose that the unknown protein spot 9 is a marker for a human cancer. Spot 9 was abundant and observed as one of three spots in a train of differing isoelectric point in one of the tear samples and weakly present in the other.

The second new protein was represented in at least 5 isoforms of different pI. Two of these were N-terminally sequenced and gave the sequence commencing EDASS (EDASSDSTGA DPAQ(E/Q)AGTSQ PNEDIAG) (spots 1 & 2).

TABLE 2

Identification of Human Reflex Tears proteins
by N-terminal sequence tagging and amino acid analysis

| | | | | |
|---|---|---|---|---|
| Spot No. | pI | Mw (k) | Sequence Tag | Identification |

| | | | | |
|---|---|---|---|---|
| 1 | 5.0 | 25 | EDASSDSTGA | unknown |
| | | | DPAQ(E/Q)AGTSQ | |
| | | | PNEDIAG | |
| 2 | 4.4 | 25 | EDASS | As for No. 1 |
| 3 | 4.6 | 14 | SSSKE | HuMan Cystatin S |
| 4 | 4.8 | 14 | SSSKE | As for No. 3 |
| 5 | 5.2 | 40 | N-terminally blocked* | Human Zn-alpha-2-glycoprotein |
| 9 | 5.1 | 10 | ¹DSGCKLLEDMVEK | Similarity with Human Mammaglobin & Rat |
| | | | | Steroid-binding proteins |
| 10 | 8.0 | 14 | WDPKE | Unknown |
| 11 | 5.1 | 18 | HHLLASDEE | Human Von Ebner's Gland Protein |
| 12 | 5.3 | 18 | \hat{ }SDEE | Von Ebner's Gland Protein |
| 14 | 8.5 | 80 | GRRR | Human lactotransferrin |

*Identified on the basis of amino acid composition matching using the AACompIdent2 program
in ExPASy (Expert Protein Analysis System) on the world wide web at
http://expasy.hcuge.cb/ch2d/aacomp2.html.
¹74 Residues sequenced
••••••••·10•••• •••••20•••••••••30••••••• ••40••••• ••••50•••••••• •60••••••• •70••••
DSGCKLLEDM VEKTINSDIS IPEYKELLQE FIDSDAAAEA MGKFKQCFLN QSHRTLKNFG LMMHTVYDSI WCNL

While the identification of spot 1 protein remains unknown it is interesting that these first N-terminal amino acids showed some sequence similarity (>40% sequence identity) to the N-terminal regions of the rat huntingtin associated protein (Swiss-Prot P54256, a protein associated with Huntington's disease and hence a possible disease marker) and the Californian sea hare Synaptotagmin (Swiss Prot P41823, a protein which binds acidic phospholipids and is possibly relevant to tear activity in phospholipid binding).

The third new protein WDPKE (spot 10) has no related proteins in the database.

By examination of the trains of spots it was possible to identify changes in the post-translational modification status of some proteins between the two samples of tears.

The results presented have identified three new proteins, one of which is related to known cancer markers, and another is differentially processed protein in tears. The present inventors have found differences in levels of the respective proteins as well as in their modification status. These discoveries indicate new targets for development of diagnostic reagents for assessing disease status. The present invention is an important new approach to the development of diagnosis as it is non-invasive and applicable to not only humans but also animals.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1-12. (canceled)

13. A method of screening for, or detecting cancer in, an animal comprising:
(a) obtaining a tear sample from the animal; and
(b) detecting the presence of a protein having a molecular weight of about 10 kiloDaltons and a pI of about 5.1 in the tear sample; wherein the presence of the protein is indicative of predisposition to, or the presence of, cancer.

14. The method according to claim 13 wherein the protein is detected by electrophoresis.

15. The method according to claim 13 wherein the electrophoresis is two-dimensional gel electrophoresis.

16. The method according to any one of claims 13 to 15 wherein the protein is detected by a labeled probe which binds to the protein.

17. The method according to claim 16 wherein the probe binds to the N-terminus of the protein.

18. The method according to claim 17 wherein the N-terminal amino acid sequence of the protein comprises SEQ ID NO: 3.

19. The method according to claim 16 wherein the probe is an antibody.

20. The method according to claim 19 wherein the antibody specifically binds to the protein.

21. The method according to claim 13 wherein the cancer is breast or prostate cancer.

22. The method according to claim 13 wherein the N-terminal amino acid sequence of the protein comprises SEQ ID NO: 3.

23. The method according to claim 13 wherein the protein is isolated from the tear sample.

24. The method according to claim 23 further comprising sequencing the N-terminal amino acids of the isolated protein.

25. The method according to claim 24 wherein the N-terminal amino acid sequence of the protein comprises SEQ ID NO: 3.

26. A protein isolated by the method of claim 24.

27. A protein isolated from tears and having a molecular weight of about 10 kiloDaltons, a pI of about 5.1 and an N-terminal amino acid sequence comprising SEQ ID NO: 3.
